(19) United States
(12) Patent Application Publication
(10) Pub. No.: US 2005/0090813 A1
(43) Pub. Date: Apr. 28, 2005

(54) Method for removing waste products produced while stripping material in transparent objects by laser-induced plasma formation

(76) Inventors: Dietrich Schweitzer, Neustadt/Orla (DE); Martin Hammer, Jena (DE)

Correspondence Address: Douglas J Christensen;Patterson Thuente Skaar & Christensen, 4800 IDS Center, 80 South 8th Street, Minneapolis, MN, 55402, US

(21) Appl. No.: 10/498,395

(22) PCT Filed: Dec. 11, 2002
(86) PCT No.: PCT/EP02/14055

(30) Foreign Application Priority Data
Dec. 12, 2001 (DE) 101-62-166.3

(51) Int. Cl. A61B 18/20

(52) U.S. Cl. 606/5

(57) ABSTRACT

Method for the removal of waste products generated while removing materials in transparent objects by way of laser-induced breakdown. The task was to quickly and safely remove the solid, liquid or gaseous waste products generated inside the object during the treatment of the material by laser. According to the invention, for the free or forced exit from the object of the waste products generated through breakdown, at least one canal, preferably of a capillary diameter, is created or opened inside the object between the ablation area of the laser material treatment and the object's exterior. The method is used in laser-induced material treatment inside of transparent objects, in particular during the intrastromal ablation of tissues of the cornea of the eye for defective vision correction.

### FIELD OF THE INVENTION

The invention covers a method for the removal of waste products generated while removing materials in transparent objects by way of laser-induced breakdown, in particular during the intrastromal ablation of tissues of the cornea of the eye for defective vision correction.

### STATE OF THE ART

It is generally known to remove material through laser-induced breakdown in order to work on objects, among them the cornea of the eye, in terms of their shape (T. Juhasz, F. H. Loesel, R. M. Kurtz et at “Corneal Refractive Surgery with Femtosecond Lasers,” IEEE J. Select. Topics Quantum Electron. 5 (4), 2000, 902910; K. R. Sletten, K. G. Yen, S. Sayegh et al.

“An in vivo model of femtosecond laser intrastromal refractive surgery,” Ophthalmic Surg Lasers 30 (9), 1999, 742-9; H. Lubatschowski, G. Maatz, A. Heisterkamp et al. “Application of ultrashort laser pulses for intrastromal refractive surgery,” Graefes Arch Clin. Exp. Ophthalmol. 238 (1), 2000, 33-9; G. Maatz, A. Heisterkamp, H. Lubatschowski et al. “Chemical and physical side effects at application of ultrashort laser pulses for intrastromal refractive surgery,” J. Opt. A: Pure Appl. Opt 2, 2000, 59-64; A. Vogel, J. Noack, K. Hahen et at “Laser-induced breakdown in the eye at pulse durations from 80 ns to 100 fs,” SPIE-Proceedings, Vol. 3255, 1998, “Application of ultrashort pulse lasers in Medicine and Biology” pp. 34-48. The said removal of material, especially from the surface of the cornea of the eye, by way of a laser is not without pain, leaves behind localized in homogeneities on the treated, optically active surface, and may lead to changes in the refractive properties of the cornea as a result of partial scar tissue formation. It is still uncertain whether the removed or locally folded-back cornea epithelium is completely regenerated and resumes its original position. In order to avoid or limit such risks, the surface area of the cornea is normally cut open, for example in a three-quarter circle, and folded back for the purpose of the intended ablation of material by laser on the inside of the cornea. After the material is removed, the folded-back surface is returned to the original position. This makes it possible to change the thickness and curvature of the cornea without at least mechanically interfering with the exterior of the cornea in the mid-section of the eye. Nevertheless, the cut into the cornea leaves permanent, large-scale damage. Errors in folding back the flaps significantly determine the quality of the optical correction that can be achieved. Cutting into the cornea changes its mechanical properties very much. This could lead to measurement values for the intraocular pressure via the normal applanation tonometry that are totally wrong. C. Knapp, H. Mittelviefhaus in their poster [sic]: “Near-blindness after LASIK” at the 99^{th}.

Conference of the German Ophthalmologic Association, Berlin Sep. 29-Oct. 2, 2001 referred to a case in which glaucoma was thereby overlooked, leading to massive eye damage.

When working on transparent materials, the suggestion has already also been made to focus the laser not on the surface but deep into the object to be worked on, and to ablate the material in the interior by way of a breakdown (plasma formation). The problem in this case, however, is the removal of the waste products generated during the recombination and condensation of the plasma. Sletten et al. (K. R. Sletten, K. G. Yen, S. Sayegh et al. “An in vivo model of femtosecond laser intrastromal refractive surgery,” Ophthalmic Surg Lasers 30 (9), 1999, 742-9) demonstrated the basic possibility to change the thickness and curvature radius of the corneas of rabbits by intrastromally applying femtosecond laser pulses. However, the removal of the waste products by diffusion assumed by these authors is neither complete nor reliably quantitatively predictable. The result is a shape of the object that is unstable over a long period and for this reason alone can hardly be considered for the treatment of the cornea of the eye. However, for changing the shape of the cornea of the eye, another approach is based on the need to cut the surface of the cornea open again (for example in a three-quarter circle) and to remove the material ablated mechanically with the laser. But even here the disadvantage is large-scale damage to the cornea.

It is generally known to suction off the smoke created by the surgical laser application. However, this can only be accomplished externally near the treated tissue. It does not allow for the extraction of the waste products from the inside of the treated object.

In summary, it can be said that the current state of the art does not allow for the defined removal of waste products generated during laser treatment directly from the inside of the treated object, or else that one must accept the disadvantage of large-scale damage in the process.

### DESCRIPTION OF THE INVENTION

The purpose of the invention is therefore to remove from the object all waste products generated during the laser-induced breakdown in transparent objects as quickly and safely as possible.

The invention creates or opens at least one canal, preferably of a capillary diameter, between the ablation area of the material treated by laser inside the object and its exterior, to ensure a free or forced exit from the object of the solid, liquid or gaseous waste products generated in connection with the breakdown. This means that at least one small canal of only capillary dimensions affecting the object or its surface is consciously accepted, in order to extract from inside the object said waste products generated during the breakdown, without making further mechanical damage to the object necessary, which is particularly important for the cornea of the eye. This relatively minor interference with the object, caused for example by a laser beam or by a capillary mechanical incision, is normally manageable in such a way that, contrary to the unavoidable serious damages to the object such as the cornea incisions described above, it is relatively negligible for the functioning of the treated objects. This now opens the possibility to carry out the suggested laser-induced material treatment, which for practical reasons is not used for said reasons, now in reproducible fashion inside transparent objects, without causing large-scale damage to the objects or having to leave them in a state of instability for long periods. For the treatment of the cornea of the eye this means, specifically, that there is no need to do mechanical work on the surface of the cornea in order to change its shape and that a certain material ablation is possible in the cornea itself, by breaking down the material to be removed, whereby the waste products exit freely or by force, without having the need to cut open large areas of the cornea. This capillary canal, of which there should be at least one, could, for example, be arranged on the periphery in such a way that it does not affect the vision of the treated eye or at least does not affect it noticeably. It has been shown that a minimum of one effluent canal of said small dimensions is sufficient for removing the waste products. A forced exit of the waste products can, for example, be reinforced by mechanical pressure on the cornea. Other possibilities are e.g. the use of suction and rinse systems or also of expulsion gas or a combination thereof.

It is of particular advantage to provide, aside from the one canal as a minimum for removing the waste products, at least one other canal (also of small dimensions) to introduce agents such as expulsion gas or rinse fluid into the ablation area of the object.

If said agents are introduced via such a canal before laying or opening at least one effluent canal, there is an increase in pressure inside the treated object inducing the combination of individual liquid or gaseous waste products such as localized gas bubbles into one uniform volume in the ablation area of the object and thereby improving or facilitating the almost total removal of the waste products after the one effluent canal as a minimum has been created or opened. Also, because of the increased pressure and depending on the property (elasticity) of the treated object, the tissues of the latter may expand with the result that, after termination of the pressure effect caused by the agent, there is also a mechanical effect exerted by the treated object itself for expelling the remaining waste products, which also furthers the removal.

It is also possible to make the at least one effluent canal discontinuous, but leaving a diffusion section through which the waste products diffuse toward the exterior for their removal.

### BRIEF DESCRIPTION OF THE DRAWING

The invention is explained in more detail using an exemplary embodiment as shown in the drawing.

FIG. 1 shows the removal of waste products during intrastromal plasma generation in the cornea of the eye by introducing agents under pressure and expulsion of the agents including the waste products.

### DETAILED DESCRIPTION OF THE DRAWING

During the intrastromal treatment of the cornea 1 of the eye, the cornea is first flattened with a pressure plate 2 to obtain a defined focal surface for a focus lens 3 through which the pulses of a treatment laser 4 in the stroma of the cornea 1 generate a plasma using a radiation strength exceeding 10¹⁰ W/cm², whereby ablation bubbles 5 are generated.

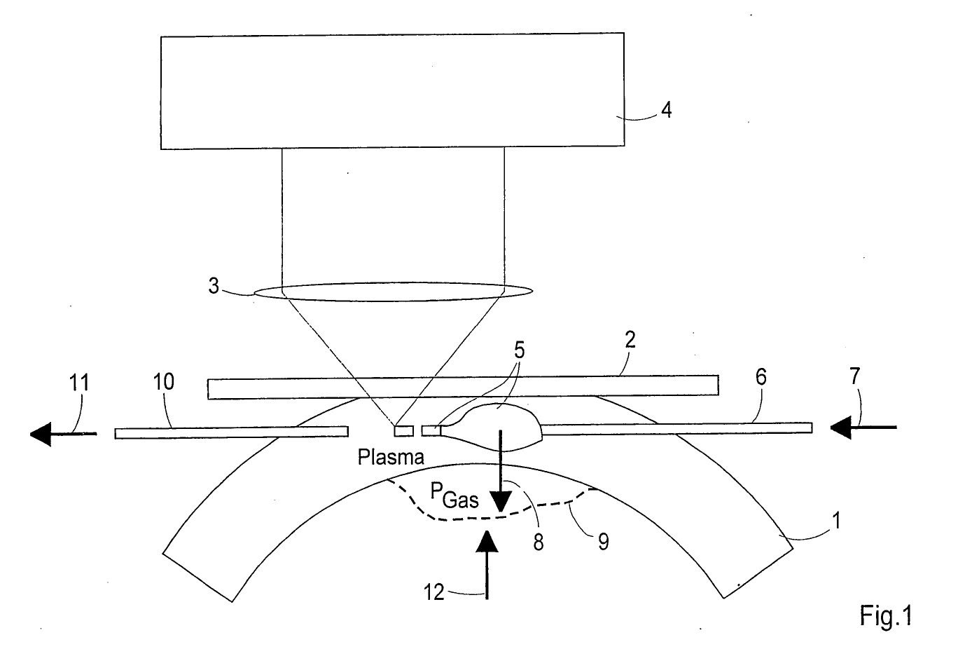

An influent canal 6 to the ablation bubbles 5 is created by way of an appropriate means, for example a capillary mechanical incision, through which (symbolized in FIG. 1 by an arrow 7) an expulsion gas is forced into the area of the cornea where the breakdown has taken place. There, the expulsion gas brings about a combination of the individual ablation bubbles 5 by tearing open the still remaining cell connections. Because of the pressure (PGas) of the expulsion gas symbolized in FIG. 1 by an arrow 8, the cavity created by the combination of the ablation bubbles 5 and the cornea 1 (the change in the shape of the cornea is indicated by a dotted line 9) expand during treatment until the growing cavity volume reaches the end of an effluent canal 10 that was introduced into the cornea 1 before the treatment, for example also by means of a capillary mechanical incision.

From this moment on, the forced-in expulsion gas, including the waste products generated during the laser treatment, exit through this effluent canal 10 (arrow 11). After turning off the force-in pressure (expulsion gas), the elasticity of the cornea 1 in connection with the intraocular pressure of the eye (symbolized in FIG. 1 by an arrow 12) facilitates the continued expulsion of the waste products through the effluent canal 10.

1. Method for the removal of waste products generated while removing materials in transparent objects by way of a laser-induced breakdown, in particular when correcting defective vision of the eye, wherein a laser is focused on the material to be removed at a chosen depth of the object and there generates certain material ablations through precisely localized plasma generation, characterized in that at least one canal, preferably of a capillary diameter, is created or opened between the ablation area and the exterior of the object for a free or forced exit from the object of the solid, liquid or gaseous waste products.

2. Method according to claim 1, characterized in that at least one canal is created or opened through the effect of laser radiation, preferably of a femtosecond laser.

3. Method according to claim 1, characterized in that the one canal as a minimum is created or opened by way of mechanical incision such as by puncturing or cutting.

4. Method according to claim 1, characterized in that the solid, liquid or gaseous waste products are discharged from the ablation area of the object via the one canal as a minimum through a free exit.

5. Method according to claim 1, characterized in that the solid, liquid or gaseous waste products are discharged from the ablation area of the object via the one canal as a minimum, by means of a forced exit.

6. Method according to claim 5, characterized in that the forced exit is effected by exerting mechanical pressure on the object.

7. Method according to claim 5, characterized in that the forced exit is effected through a suction device connected to, or introduced into, the one canal as a minimum.

8. Method according to claim 5, characterized in that the forced exit is effected through a rinsing device connected to, or introduced into, the one canal as a minimum.

9. Method according to claim 5, characterized in that the forced exit is effected using expulsion gas.

10. Method according to claim 5, characterized in that the forced exit is effected by using several of the methods described in claims 6 to 9 for the removal of the waste products.

11. Method according to claim 5, characterized in that several canals are created or opened, wherein at least one canal is used for the removal of solid, liquid or gaseous waste products and at least one further canal is used for introducing agents such as expulsion gas or rinse fluid into the ablation area of the object, for the forced exit of the waste products.

12. Method according to claim 11, characterized in that in order to increase pressure in the ablation area of the object in which the waste products are or will be generated during the laser treatment, and, as the case may be, for the purpose of the elastic expansion of the ablation area, initially at least one further canal for introducing the agent or agents and, only after combining the individual waste products generated locally in the ablation area, the one canal at least for their joint and complete removal are created or opened.

13. Method according to claim 11, characterized in that in order to increase pressure in the ablation area of the object, in which the waste products are or will be generated during the laser treatment, and, as the case may be, for the purpose of the elastic expansion of the ablation area, the agent or agents are introduced under pressure.

14. Method according to claim 1, characterized in that the one canal as a minimum is not created as a continuous canal but leaves a diffusion section through which the waste products diffuse toward the exterior for their removal.
